(19) United States
(12) Patent Application Publication
(10) Pub. No.: US 2008/0114230 A1
(43) Pub. Date: May 15, 2008

(54) ELECTRODE SUPPORT

(76) Inventor: Bruce Addis, Redwood City, CA (US)

Correspondence Address: BOZICEVIC, FIELD & FRANCIS LLP;(PROTEUS BIOMEDICAL, INC), 1900 UNIVERSITY AVENUE, SUITE 200, EAST PALO ALTO, CA, 94303, US

(21) Appl. No.: 11/939,524

(22) Filed: Nov. 13, 2007

Related U.S. Application Data
(60) Provisional application No. 60/865,760, filed on Nov. 14, 2006.

(51) Int. Cl. A61N 1/05

(52) U.S. Cl. 600/373; 607/116

(57) ABSTRACT

Electrode supports configured to provide a foundation for a segmented electrode on a flexible lead structure are provided. Also provided are electrode structures, leads that include the same, implantable pulse generators that include the leads, as well as systems and kits having components thereof, and methods of making and using the subject devices.

### CROSS REFERENCES TO RELATED APPLICATIONS

Pursuant to 35 U.S.C. § 119 (e), this application claims priority to U.S. Provisional Application Ser. No. 60/865,760 filed Nov. 14, 2006; the disclosure of which priority application is herein incorporated by reference.

### INTRODUCTION

Pacemakers and other implantable medical devices find wide-spread use in today's health care system. A typical pacemaker includes stimulating electrodes that are placed in contact with heart muscle, detection electrodes placed to detect movement of the heart muscle, and control circuitry for operating the stimulating electrodes based on signals received from the detection electrodes. Thus, the pacemaker can detect abnormal (e.g., irregular) movement and deliver electrical pulses to the heart to restore normal movement.

Pacing leads implanted in vessels in the body are, for many applications, flexible cylindrical devices. They are cylindrical due to three main reasons: most anatomical conduits are cylindrical, medical sealing and access devices seal on cylindrical shapes and cylindrical leads have uniform bending moments of inertia around the long axis of the device. The cylindrical nature of the device necessitates the cylindrical design of pacing electrodes on the body of the device.

Due to the tortuous nature of the vessels in the body, following implantation the rotational orientation of one electrode can not be predetermined in many currently employed devices. As such, many currently employed lead devices employ cylindrical electrode designs that are conductive to tissue around the entirety of the diameter of the lead. This insures that some portion of the cylindrical electrode contacts excitable tissue when they are implanted. Despite the multiple devices in which cylindrical continuous ring electrodes are employed, there are disadvantages to such structures, including but not limited to: undesirable excitation of non-target tissue, e.g., which can cause unwanted side effects, increased power use, etc.

An innovative way to address this problem is to employ segmented electrode structure, in which the circular band electrode is replaced by an electrode structure made up of two or more individually activatible and electrically isolated electrode structures that are configured in a discontinuous band. Such segmented electrode structures are disclosed in published PCT application Publication Nos. WO 2006/069322 and WO2006/029090; the disclosures of which are herein incorporated by reference.

While providing significant improvements in functionality, segmented electrode structures can lack structural robustness that is sufficient for certain applications. Accordingly, there is continued interest in the development of improved segmented electrode structures which are more structurally robust.

### SUMMARY

The present invention provides significantly improved electrode structures, including segmented electrode structures, which are robust and able to withstand a variety of different stress inducing conditions when implanted into a patient. As such, the present invention provides implantable devices that include satellite electrodes which can be implanted and maintain performance for long periods of time.

Embodiments of the invention include electrode supports configured to provide a foundation for a segmented electrode on a flexible medical carrier, e.g., vascular lead, structure. Also provided are satellite electrode structures, leads that include the same, implantable pulse generators that include the leads, as well as systems and kits having components thereof, and methods of making and using the subject devices.

### BRIEF DESCRIPTION OF THE FIGURES

FIG. 1 provides a three-dimensional view of an electrode support in accordance with an embodiment of the invention;

FIG. 2 provides a cross-sectional view of the structure shown in FIG. 1;

FIG. 3 provides a more detailed cross sectional view of a portion of the structure shown in FIG. 1 illustrating the overhang adjacent to the recess which provides for secure fitting of an electrode on the surface of the support;

FIG. 4 provides a three-dimensional view of a support/electrode structure according to an embodiment of the invention that includes a support as shown in FIG. 1 and planar electrodes positioned in the recesses of the support;

FIG. 5A provides a three-dimensional view of a hermetically sealed control integrated circuit (IC) chip that is positioned inside of a support according to embodiments of the invention;

FIG. 5B illustrates how the chip shown in FIG. 5A may be positioned inside of the support structure shown in FIG. 1;

FIG. 5C illustrates a support/chip structure where the chip shown in FIG. 5A has been positioned inside of the support structure shown in FIG. 1;

FIG. 5D provides a three dimension transparent view of the structure shown in FIG. 5C;

FIG. 6 illustrates an exemplary external view of a number of pacing satellites, in accordance with an embodiment of the present invention.

FIG. 7A provides a three-dimensional view of satellite electrode structure of the invention as positioned relative to the conductive members of a lead, according to an embodiment of the invention;

FIG. 7B provides a cross-sectional view of the satellite electrode structure shown FIG. 7A; and

FIG. 8 provides a depiction of a cardiac resynchronization therapy system that includes one or more hermetically sealed integrated circuits coupled to lead electrodes according to an embodiment of the invention.

### DETAILED DESCRIPTION

As summarized above, the present invention provides significantly improved satellite electrode structures, including segmented electrode structures, which are robust and able to withstand a variety of different stress inducing conditions when implanted into a patient. As such, the present invention provides implantable devices that include satellite electrodes which can be implanted and maintain performance for long periods of time. Embodiments of the invention include electrode satellite supports configured to provide support for a segmented electrode on a flexible medical carrier, e.g., vascular lead, structure. Also provided are satellite electrode structures, leads that include the same, implantable pulse generators that include the leads, as well as systems and kits having components thereof, and methods of making and using the subject devices.

In further describing aspects of the invention in greater detail, embodiments of the electrode support structures are reviewed first in greater detail. Next, a review of electrodes that include the support structures, as well as medical carriers and medical devices that include the same is provided. In addition, a further description of kits and systems of the invention, and methods of using various aspects of the invention, is provided.

### Electrode Support Structures

As summarized above, the invention provides electrode supports. By “electrode support” is meant a structure or object that provides a foundation for electrodes that may be present on an implanted medical device. As such, the structure serves to maintain the position of electrodes associated therewith in three dimensional space. In certain embodiments, the structure is configured to be a satellite electrode support structure. As used herein, the term satellite electrode refers to a device that includes one or more electrodes positioned in the body some distance away from a control element that controls the functionality of the electrode, where communication between the control element and the satellite electrode may be by one or more wires or wireless.

Electrode support structures in accordance with embodiments of the invention include a tubular base support having an inner and an outer surface; and at least one recess on said outer surface configured to receive an electrode inset. The term “tubular” is used broadly to refer to a hollow body having an outer surface and an inner surface. The inner cross sectional shape of the tubular based support may vary greatly. Configurations include, but are not limited to: rectangular, square, rhombic, triangular or V-shaped, D-shaped, U-shaped, circular, semicircular, ellipsoid, a portion, e.g., half, that can be combined with other portions to make whole support structure, and the like. In certain embodiments, the tubular base support has a circular cross section shape and therefore the support may be described as a hollow cylinder. Depending on the particular application for which the support is designed, the dimensions of the support may vary. In certain embodiments, e.g., where the support is present on a vascular lead, the support may have: an outer diameter ranging from about 0.25 to about 40 mm, such as from about 1 to about 10 mm and including from about 1 to about 4 mm; an inner diameter ranging from about 0.1 to about 35 mm, such as from about 0.5 to about 8 mm and including from about 0.5 to about 3.5 mm; and a length ranging from about 0.5 to about 50 mm, such as from about 1 mm to about 20 mm and including from about 1 mm to about 5 mm. In certain embodiments, the base support has a length that is longer than its width, e.g., by a factor of about 1.5 or more, such as about 2.5 or more, e.g., about 5 or more.

Present on the outer surface of the support is at least one recess, where the recess is configured for holding an electrode structure in a manner such that the electrode structure is securely coupled to the recess. By securely coupled is meant that the electrode does not readily dissociate from the recess and support under conditions of its intended use, e.g., when implanted in at a cardiovascular location and subjected to forces found at such a site. In certain embodiments, the recess is bounded on at least one side, and certain embodiments on two opposing sides, by raised structure(s) having an overhang (i.e., lip) to secure an electrode in the recess. The raised structure may, in certain embodiments, have a height as measured relative to the bottom of the recess, ranging from about 0.025 to about 1 mm, such as from about 0.025 to about 0.25 mm. In certain embodiments, the overhang or lip may extend beyond the plane of the raised structure and over the recess by a distance ranging from about 0.025 to about 3.5 mm, such as from about 0.025 to about 0.25 mm.

In certain embodiments, the supports are configured for use in segmented electrode structures. By segmented electrode structure is meant an electrode structure that includes two or more, e.g., three or more, including four or more, disparate electrode elements. Embodiments of segmented electrode structures are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various segmented electrode structures of these applications being herein incorporated by reference. In these embodiments, the support may include a recess for each electrode element of the segmented electrode structure. As such, the support may include 2 or more, 3 or more, 4 or more, etc., where each recess is configured to receive an electrode element (i.e., an electrode inset).

In certain embodiments, the structures are configured for use as supports for “addressable” electrode structures. Addressable electrode structures include structures having one or more electrode elements directly coupled to control circuitry, e.g., present on an integrated circuit (IC). Addressable electrode structures include satellite structures that include one more electrode elements directly coupled to an IC and configured to be placed along a lead. Examples of addressable electrode structures that include an IC are disclosed in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various addressable electrode structures of these applications being herein incorporated by reference. In these embodiments where the supports are configured to support an addressable electrode structure that includes an IC, the support may include IC holding elements that immobilize an IC inside the support. IC holding elements of interest include, but are not limited clamps, clips, notches configured to receive a portion (e.g., edge) of an IC, etc.

The base support structure can be fabricated from any convenient material having sufficient hardness to provide desired functionality in its intended functionality. In certain embodiments, the support is fabricated from a dielectric material, where such materials include, but are not limited to: ceramics, e.g., alumina, polymers, and the like; crystals, e.g., silicon, quartz; metals, with dielectric coatings, etc.

The support may be fabricated using any convenient fabrication protocol. Protocols of interest may include one or more of: extruding, pressing, machining, molding, cutting, etc. For example, the support can be extruded and cut, molded, micromachined, cut from a sheet, e.g., via a laser or stamping protocol, or combinations thereof.

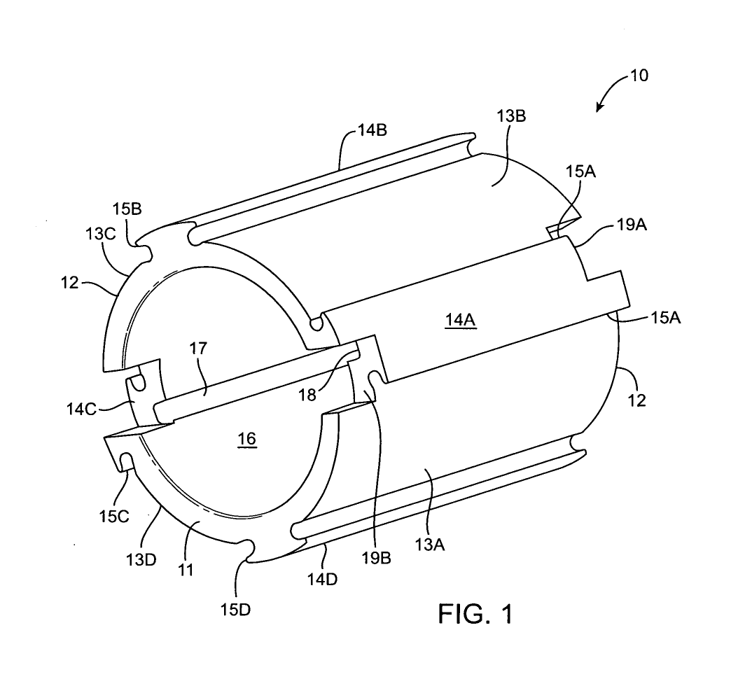

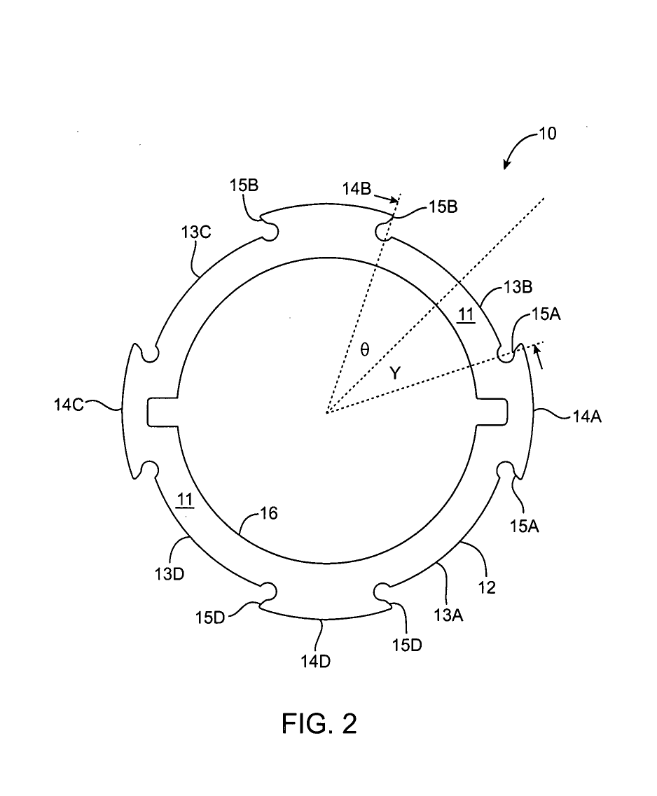

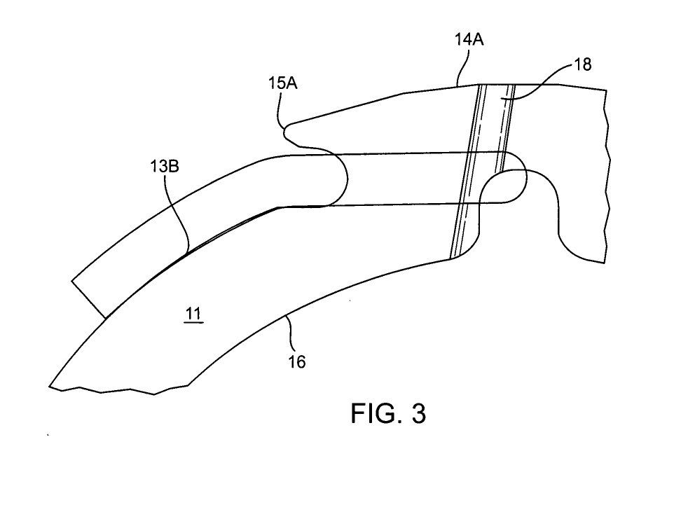

FIG. 1 provides a three dimensional view of an implantable electrode support according to an embodiment of the invention. In FIG. 1, support 10 includes a hollow cylindrical base support 11 having a length longer than its inner diameter. Outer surface 12 includes four distinct recesses 13A, 13B, 13C, 13D each configured to receive an electrode element, such as the petal electrode elements described in PCT/US2005/046811 titled “Implantable Addressable Segmented Electrodes” filed on Dec. 22, 2005 the description of which petal electrodes is incorporated herein by reference. Separating each recess is a raised structure 14A, 14B, 14C and 14D that includes an overhang or lip 15A, 15B, 15C and 15D that serves to secure an electrode element in the recess. Also shown on the internal surface 16 are notches 17 and 18 configured to receive an IC stably inside the support, e.g., where opposing edges of the IC may be slid into the notches during fabrication. FIG. 2 provides a cross-sectional view of the support structure shown in FIG. 1. Angles θ and γ may vary, and in certain embodiments these angles range from about 10 to about 180°. FIG. 3 provides a close-up cross-sectional view of a portion of raised structure 14A. In this figure, overhang 15A is clearly shown to be configured to secure an electrode element that is slid into recess 13B.

### Electrode Satellite Structures

Embodiments of the invention further include electrode assemblies, such as electrode satellite structures, where the structures include an electrode support, such as those described above, and at least one electrode element. In further embodiments, the satellite structures may include control circuitry, e.g., in the form of an IC (e.g., an IC inside of the support), such that the satellite structure is addressable. In certain embodiments, the structure includes two or more electrode elements, such as three or more electrode elements, including four or more electrode elements, e.g., where the structure is a segmented electrode structure.

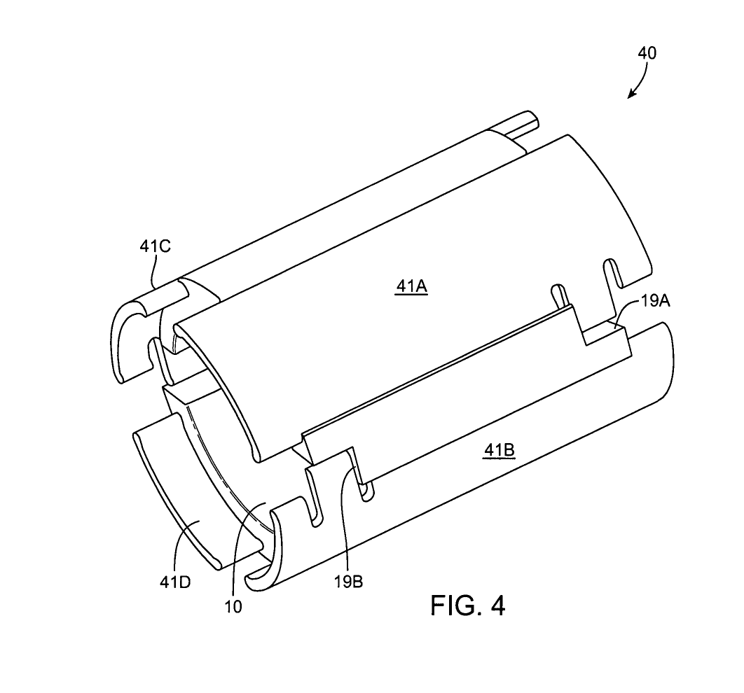

FIG. 4 provides a view of a segmented electrode structure 40 that includes a support 10 analogous to that shown in FIGS. 1 to 3 with four distinct electrode elements 41A, 41B, 41C and 41D present in the recesses of the support. Support 10 includes feed through notches 19A and 19B which provide access from the interior of the support to the electrode element. In this embodiment, the notches also serve to align the electrode elements relative to the support.

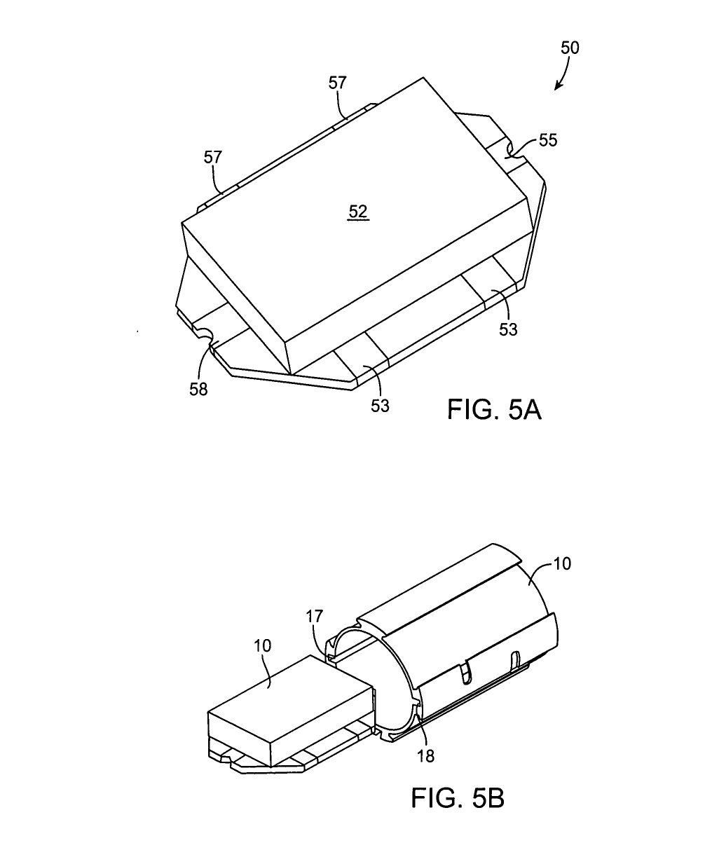

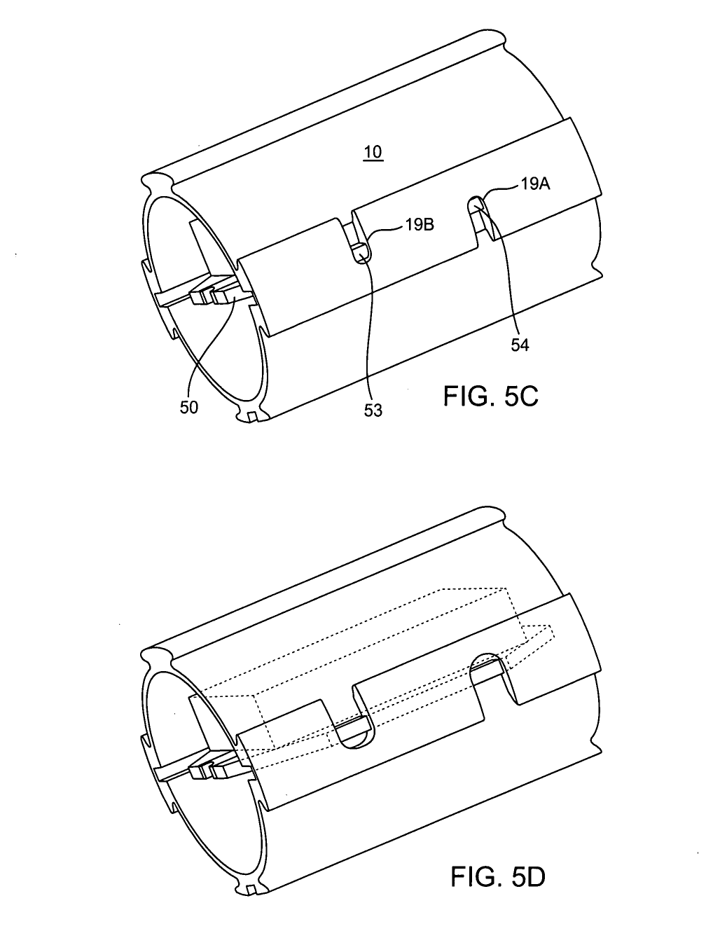

As described above, the electrode assemblies may include an IC chip or other control element which imparts addressability to the assembly. FIG. 5A provides a view of a hermetically sealed IC chip that may find use in certain embodiments of the invention. IC chip 50 is a hermetically sealed structure in which the circuitry is present in a sealed housing and is electrically accessible by conductive elements 53, 54, 55, 56, 57 and 58. Embodiments of hermetically sealed IC chips include, but are not limited to, those described in PCT application serial PCT/US2005/046815 titled “Implantable Hermetically Sealed Structures” and filed on Dec. 22, 2005, the description of hermetically sealed structures provided in this application being specifically incorporated herein by reference. FIG. 5B provides an illustration of the IC 50 being slid into the slots 17 and 18 of support 10 to produce the structure shown in FIG. 5C in which IC 50 is stably positioned inside of the support 10. Also shown are conductive elements 53 and 54 that are accessible through notches 19A and 19B of the support 10. FIG. 5D provides another transparent view of the structure shown in FIG. 5C.

As summarized above, the invention provides implantable medical devices that include the electrode structures as described above. By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human.

### Vascular Leads

Embodiments of the invention also include medical carriers that include one or more electrode satellite structures, e.g., as described above. Carriers of interest include, but are not limited to, vascular lead structures, where such structures are generally dimensioned to be implantable and are fabricated from a physiologically compatible material. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 connector, for connecting to a control unit, e.g., present in a “can” or analogous device. The lead may include one or more lumens, e.g., for use with a guidewire, for housing one or more conductive elements, e.g., wires, etc. The distal end may include a variety of different features as desired, e.g., a securing means, etc.

In certain embodiments of the subject systems, one or more sets of electrode satellites as described above are electrically coupled to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or less wires, such as only 2 wires or only 1 wire. Multiplex lead structures of interest include those described in application Ser. No. 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

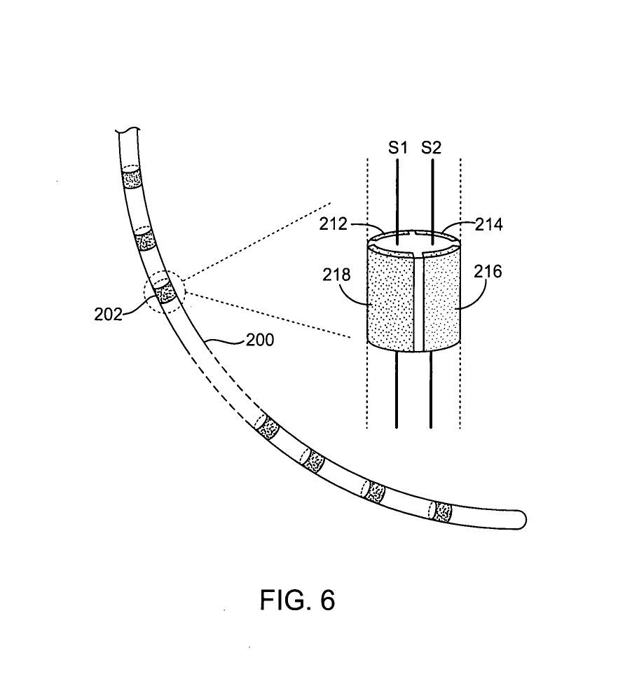

FIG. 6 illustrates an external view of a number of exemplary pacing satellites, in accordance with a multiplex lead embodiment of the present invention. According to one embodiment, a pacing lead 200 (e.g., right ventricular lead 109 or left ventricular lead 107 of FIG. 9) accommodates two bus wires S1 and S2, which are coupled to a number (e.g., eight) of satellites, such as satellite 202. FIG. 6 also shows satellite 202 with an enlarged view. Satellite 202 includes electrodes 212, 214, 216, and 218, located in the four quadrants of the cylindrical outer walls of satellite 202 and supported by a support structure of the invention. Each satellite also contains a control chip inside the structure which communicates with a pacing and signal-detection system to receive configuration signals that determine which of the four electrodes are to be coupled to bus wires S1 or S2.

The configuration signals, the subsequent pacing pulse signals, and the analog signals collected by the electrodes can all be communicated through bus wires S1 and S2, in either direction. Although shown in a symmetrical arrangement, electrodes 212, 214, 216 and 218 may be offset along lead 200 to minimize capacitive coupling among these electrodes. The quadrant arrangement of electrodes allows administering pacing current via electrodes oriented at a preferred direction, for example, away from nerves, or facing an electrode configured to sink the pacing current. Such precise pacing allows low-power pacing and minimal tissue damage caused by the pacing signal.

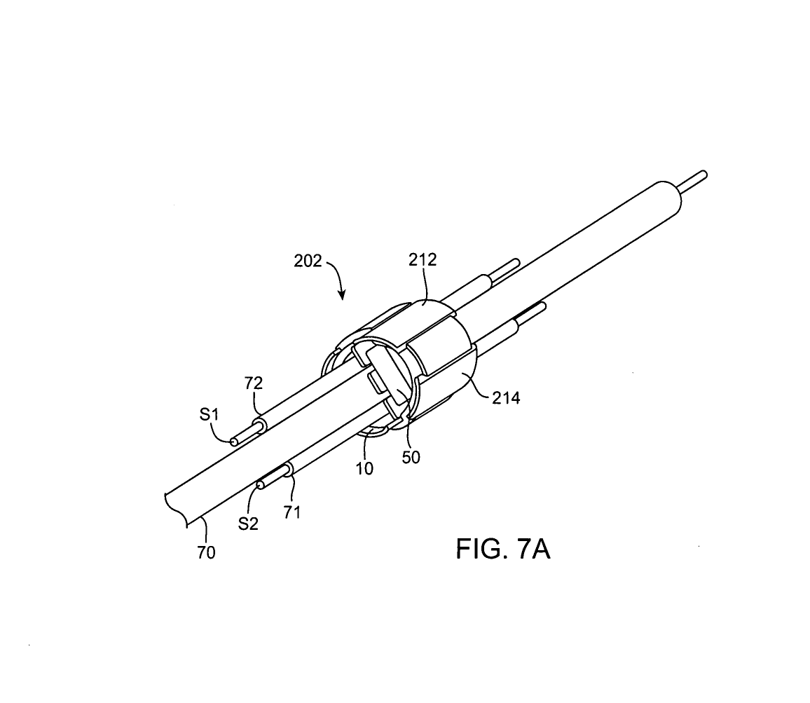

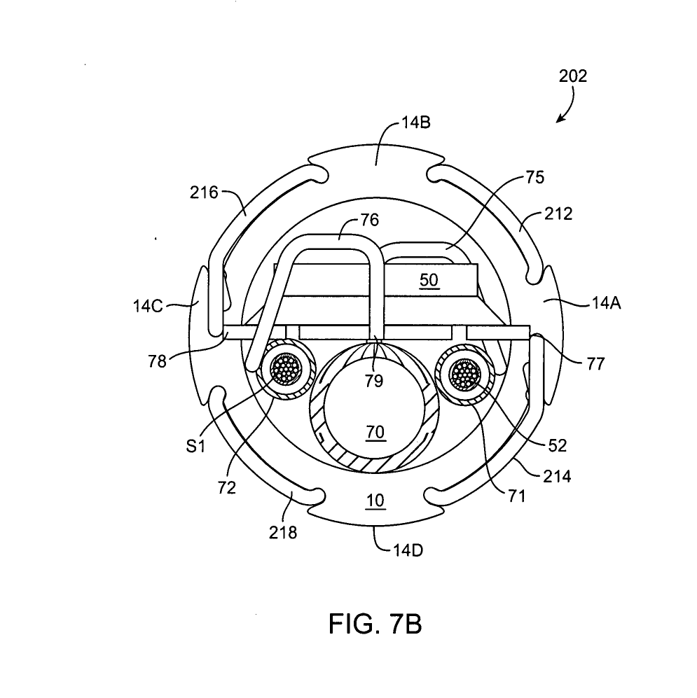

FIG. 7A provides cutaway three dimensional view of a satellite of the lead shown in FIG. 6. Shown in FIG. 7A is satellite 202 having four electrodes 212, 214, 216 and 218 present in the recesses of support structure 10. IC 15 is present inside support structure 10 and is electrically coupled to the electrodes. Also shown are the three internal lumens of the lead, that include the central guidewire lumen 70, as well as conductive element lumens 71 and 71 which hold wires S1 and S2.

FIG. 7B provides a cross-sectional view of satellite 202. Satellite 202 includes support 10 having four electrode elements 212, 214, 216 and 218 secured into its recesses and separated by raised structures 14A, 14B, 14C and 14D. Present inside of the support 10 is IC 50. IC 50 is electrically coupled to S1 in lumen 72 by flexible conductive element 76. Flexible conductive element 76 is coupled to the IC at connection point 79. Similarly, IC 50 is electrically coupled to S1 in lumen 71 by flexible conductive element 75. The electrodes are also electrically coupled to the IC 50, e.g., as illustrated by connection 77 between electrode 214 and IC 50 and connection 78 between electrode 216 and IC 50.

The leads may further include a variety of different effector element, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

### Implantable Pulse Generators

Embodiments of the invention further include implantable pulse generators. Implantable pulse generators may include: a housing which includes a power source and an electrical stimulus control element; one or more vascular leads as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

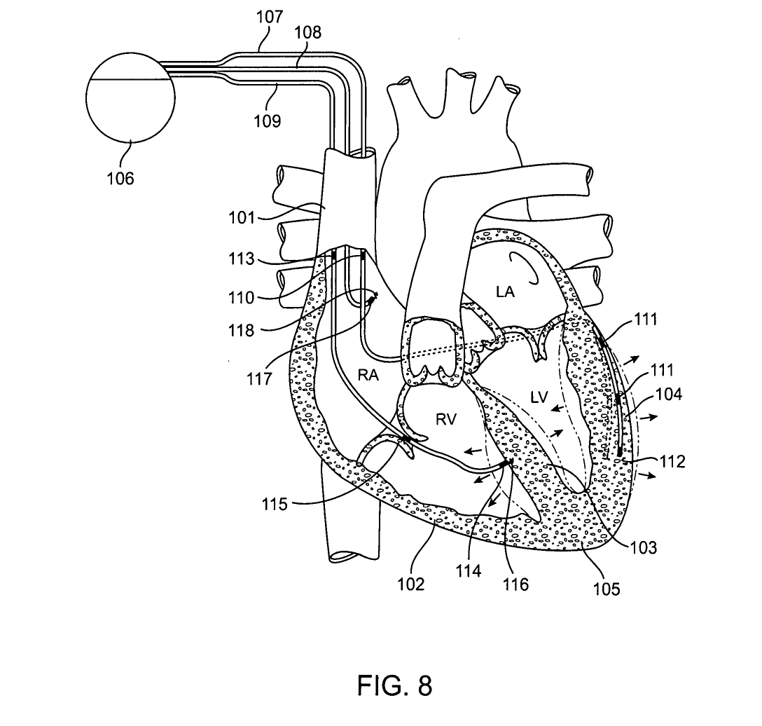

An implantable pulse generator according to an embodiment of the invention is depicted in FIG. 8, which provides a cross-sectional view of the heart with of an embodiment of a cardiac resynchronization therapy (CRT) system. The system includes a pacemaker can 106 that includes a control element (e.g., processor) and a power source, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more satellite electrode assemblies 110,111, and 112. Each of the electrodes assemblies is a satellite as described above and includes a hermetically sealed integrated circuit electrically coupled to four distinct electrode element arranged in a quadrant configuration, such as shown in FIG. 7B. Having multiple distal electrode assemblies allows a choice of optimal electrode location for CRT. In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iridium) electrode assemblies 110,111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106, e.g., via an IS-1 connector.

The electrode lead 107 is placed in the heart using standard cardiac lead placement devices which include introducers, guide catheters, guidewires, and/or stylets. Briefly, an introducer is placed into the clavicle vein. A guide catheter is placed through the introducer and used to locate the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi-electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113,114,115.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into the clavicle vein, through the superior vena cava, through the right atrium and down into the right ventricle. Electrode lead 109 is positioned under fluoroscopy into the location the clinician has determined is clinically optimal and logistically practical for fixating the electrode lead 109. Under fluoroscopy, the active fixation helix 116 is advanced and screwed into the cardiac tissue to secure electrode lead 109 onto the septum. The electrode lead 108 is placed in the right atrium using an active fixation helix 118. The distal tip electrode 118 is used to both provide pacing and motion sensing of the right atrium.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various methods of operation of these applications being herein incorporated by reference and applicable for use of the present devices.

### Systems

Also provided are systems that include one more devices as described above, an implantable pulse generator. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, on a surface of the body or separate from the body during use.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 400 to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

### Methods of Making

The subject structures and devices described herein may be fabricated using any convenient protocol. Aspects of the invention include methods of making a vascular lead electrode satellite, where the method includes providing an electrode support as described above and positioning an electrode element in a recess of the support, and in certain embodiments additionally includes placing an IC in the support such that the IC is electrically coupled to the electrode element(s) in the recess(es) of the support. In certain embodiments, the positioning step includes fitting a premade electrode element into the recess, e.g., by sliding the electrode into the recess. As such, a premade electrode element, such as a petal electrode as described in PCT/US2005/46811 titled “Implantable Addressable Segmented Electrodes” filed on Dec. 22, 2005, may be slid into the recess to produce the desired electrode structure.

In yet other embodiments, the electrode element may be positioned in the recess by depositing a conductive material into said recess. Any convenient deposition protocol may be employed. In certain embodiments, the depositing is achieved by cathodic arc deposition, where the electrode element is deposited into the recess of the support using cathodic arc plasma deposition protocols. In cathodic arc plasma deposition, a form of ion beam deposition, an electrical arc is generated between a cathode and an anode that causes ions from the cathode to be liberated from the cathode and thereby produce an ion beam. The resultant ion beam, i.e., plasma of cathodic material ions, is then contacted with a surface of a substrate (i.e., material on which the structure is to be produced) to deposit a structure on the substrate surface that is made up of the cathodic material, and in certain embodiments element(s) obtained from the atmosphere in which the substrate is present.

A number of patents and published applications are available which describe various cathodic arc deposition protocols and systems. Such publications include U.S. Pat. Nos. 6,929,727; 6,821,399; 6,770,178; 6,702,931; 6,663,755; 6,645,354; 6,608,432; 6,602,390; 6,548,817; 6,465,793; 6,465,780; 6,436,254; 6,409,898; 6,331,332; 6,319,369; 6,261,421; 6,224,726; 6,036,828; 6,031,239; 6,027,619; 6,026,763; 6,009,829; 5,972,185; 5,932,078; 5,902,462; 5,895,559; 5,518,597; 5,468,363; 5,401,543; 5,317,235; 5,282,944; 5,279,723; 5,269,896; 5,126,030; 4,936,960; and Published U.S. Application Nos.: 20050249983; 20050189218; 20050181238; 20040168637; 20040103845; 20040055538; 20040026242; 20030209424; 20020144893; 20020140334 and 20020139662. See also U.S. Provisional Application Ser. No. 60/805,464 filed Jun. 21, 2006 entitled “Implantable Medical Devices Comprising Cathodic Arc Produced Structures”; 60/805,578 filed Jun. 22, 2006 entitled “Cathodic Arc Deposition Hermetically Sealed Implantable Structures”; 60/805,581 filed Jun. 22, 2006 entitled “Noble Metal Compounds Produced By Cathodic Arc Deposition”; and 60/805,576 filed Jun. 22, 2006 entitled “Implantable Medical Devices Comprising Cathodic Arc Produced Structures”; all incorporated by reference in their entirety herein.

The cathodic arc produced electrode elements of the invention are, in certain embodiments, thick, stress-free metallic structures. In certain embodiments, the electrode elements range in thickness from about 0.01 μm to about 500 μm, such as from about 0.1 μm to about 150 μm. In certain embodiments, the structures have a thickness of about 1 μm or greater, such as a thickness of about 25 μm or greater, including a thickness of about 50 μm or greater, where the thickness may be as great at about 75, 85, 95 or 100 μm or greater. In certain embodiments, the thickness of the structures ranges from about 1 to about 200, such as from about 10 to about 100 μm.

The cathodic arc produced electrode elements are, in certain embodiments, stress-free. By “stress-free” is meant that the structures are free of defects that would impair the functionality of the structure. As such, “stress-free” means low stress as compared to stress that would case the structures to pull away, e.g., delaminate, from the substrate on which they are deposited. Accordingly, the structures are free of cracks, gaps, holes, or other defects, particularly those which would impair the function of the structure, e.g., the ability of the structure to seal an internal volume of the device, serves as a conductive element, etc.

In certain embodiments, the electrode element is a metallic layer. In certain embodiments, the metallic electrodes are structures that include a physiologically compatible metal, where physiologically compatible metals of interest include, but are not limited to: gold (Au), silver (Ag), nickel (Ni), Osmium (Os), palladium (Pd), platinum (Pt), rhodium (Rh), iridium (Ir) titanium (Ti), aluminum (Al), vanadium (V), zirconium (Zr), molybdenum (Mo), iridium (Ir), thallium (TI), tantalum (Ta), and the like. In certain embodiments, the metallic structure is a pure metallic structure of a single metal. In yet other embodiments, the metallic structure may be an alloy of a metal and one or more additional elements, e.g., with the metals listed above or other metals, e.g., chromium (Cr), tungsten (W), etc. In yet other embodiments, the structure may be a compound of a metal and additional elements, where compounds of interest include but are not limited to: carbides, oxides, nitrides, etc. Of particular interest in certain embodiments are layers that include platinum, where such layers may be pure platinum or a combination of platinum and another element. Examples of compounds of interest include binary compounds, e.g., Ptir, PtTi, TiW and the like, as well as ternary compounds, e.g., carbonitrides, etc.

### Kits

Also provided are kits that include the subject electrode structures, as part of one or more components of an implantable device or system, such as an implantable pulse generator, e.g., as reviewed above. In certain embodiments, the kits further include at least a control unit, e.g., in the form of a pacemaker can. In certain of these embodiments, the structure and control unit may be electrically coupled by an elongated conductive member. In certain embodiments, the electrode structure may be present in a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An Implantable electrode support comprising:
a tubular base support having an inner and outer surface; and
at least one recess on said outer surface configured to receive an electrode inset.

2. The implantable electrode support according to claim 1, wherein said support comprises two or more recesses on said outer surface, wherein each recess is configured to receive an electrode.

3. The implantable electrode support according to claim 2, wherein said support comprises four recesses on said outer surface, each configured to receive an electrode.

4. The implantable electrode support according to claim 1, wherein said recess is bounded on at least one side by raised structure having an overhang to secure an electrode in said recess.

5. The implantable electrode support according to claim 1, wherein said tubular base support is a cylindrical structure.

6. The implantable electrode support according to claim 1, wherein said base support has a length that is longer than its width.

7. The implantable electrode support according to claim 1, wherein said base support comprises notches on said inner surface configured to receive an integrated circuit device.

8. The implantable electrode support according to claim 1, wherein said base support comprises a material having a hardness that is substantially the same as or greater than silicon.

9. The implantable electrode support according to claim 1, wherein said base support is fabricated from a dielectric material.

10. The implantable electrode support according to claim 9, wherein said dielectric material is alumina.

11. An electrode assembly comprising:
(a) an implantable electrode support according to claim 1;
(b) an electrode present in a recess of said support; and
(c) a integrated circuit chip present inside of said support.

12. The electrode assembly according to claim 11, wherein said electrode assembly is a segmented electrode comprising two or more electrodes.

13. The electrode assembly according to claim 12, wherein said segmented electrode comprises four electrodes.

14. An elongated flexible structure comprising a proximal end and a distal end, and at least one electrode assembly according to claim 11.

15. The elongated flexible structure according to claim 14, wherein said structure is a vascular lead.

16. The elongated flexible structure according to claim 15, wherein said vascular lead comprises 2 or more electrode assemblies each comprising:
(a) an implantable electrode support comprising a tubular base support having an inner and outer surface; and at least one recess on said outer surface configured to receive an electrode inset;
(b) an electrode present in a recess of said support; and
(c) a integrated circuit chip present inside of said support.

17. The elongated flexible structure according to claim 16, wherein said vascular lead is a multiplex lead having 3 or less wires.

18. The elongated flexible structure according to claim 17, wherein said vascular lead includes only 2 wires.

19. The elongated flexible structure according to claim 17, wherein said vascular lead includes only 1 wire.

20. The elongated flexible structure according to claim 12, wherein said vascular lead includes an IS-1 connector at said proximal end.

21. An implantable pulse generator comprising:
(a) a housing comprising a power source and an electrical stimulus control element; and
(b) a vascular lead according to claim 15.

22. The implantable pulse generator according to claim 21, wherein said generator comprises two or more vascular leads.

23. The implantable pulse generator according to claim 21, wherein said control element is configured to operate said implantable pulse generator as a pacemaker.

24. The implantable pulse generator according to claim 21, wherein said control element is configured to operate said implantable pulse generator in a manner sufficient to achieve cardiac resynchronization.

25. A method of making a vascular lead electrode satellite, said method comprising:
(a) providing an electrode support according to claim 1; and
(b) positioning an electrode in said recess of said support.

26. The method according to claim 25, wherein positioning comprises fitting said electrode into said recess.

27. The method according to claim 26, wherein said electrode is fit into said recess by sliding said electrode into said recess.

28. The method according to claim 25, wherein said electrode is positioned into said recess by depositing a conductive material into said recess.

29. The method according to claim 28, wherein said depositing is by cathodic arc deposition.

30. The method according to claim 25, wherein said electrode comprises platinum.

31. A system comprising:
(a) a first implantable pulse generator according to claim 21; and
(b) a second device configured to communicate with said implantable pulse generator.

32. The system according to claim 31, wherein said second device is an implantable medical device.

33. A method comprising:
implanting an implantable pulse generator according to claim 21 into a subject; and
using said implanted pulse generator.

34. The method according to claim 33, wherein said using comprises activating at least one of said electrodes of said pulse generator to deliver electrical energy to said subject.

35. The method according to claim 34, wherein said method further comprises determining which of the electrodes of said pulse generator to activate.

36. A kit comprising:
(a) a housing comprising a power source and an electrical stimulus control element; and
(b) a vascular lead according to claim 15.
